(19) United States
(12) Patent Application Publication
(10) Pub. No.: US 2024/0318150 A1
(43) Pub. Date: Sep. 26, 2024

(54) METHODS FOR OXYFUNCTIONALIZATION OF VARIOUS SUBSTRATES USING BACTERIAL ENZYMES

(71) Applicant: GECCO Biotech B.V., Groningen (NL)

(72) Inventors: Nikola LONCAR, Groningen (NL); Hugo VAN BEEK, Groningen (NL); Marco Wilhelmus FRAAIJE, Groningen (NL)

(73) Assignee: GECCO Biotech B.V., Groningen (NL)

(21) Appl. No.: 18/580,343

(22) PCT Filed: Jul. 20, 2022
(86) PCT No.: PCT/NL2022/050425

§ 371 Date: Jan. 18, 2024

(30) Foreign Application Priority Data
Jul. 20, 2021 (EP) 21186631.4

(51) Int. Cl. C12N 9/08; C12P 17/16

(52) U.S. Cl. CPC C12N 9/0065; C12P 17/165; C12Y 111/02001

(57) ABSTRACT

The invention relates to the field of protein engineering and biocatalysis, in particular to methods for oxygenation of aliphatic alkenes and terpenes using bacterial enzymes. Provided is a method for oxyfunctionalization of a substrate of interest, comprising contacting an aliphatic alkene or a terpene substrate with a source of hydrogen peroxide and a polypeptide having calcosin-like peroxy genase activity (EC 1.11.2.1), wherein the polypeptide is selected from the group consisting of: (a) a polypeptide comprising an amino acid sequence having at least 50% pairwise sequence identity when aligned to at least 150 consecutive amino acid residues of Seq. No. 2 shown in Table 1, and comprising at least the following heme-coordinating motifs: i) HXXFFD: ii) H(X)XD, wherein X is any amino acid; and (b) a fragment of the polypeptide of (a) that has calcosin-like peroxygenase activity.

The invention relates to the field of protein engineering and biocatalysis. More in particular, it relates to novel polypeptides capable of oxygenation of, among others, cyclic and non-cyclic aliphatic alkenes, terpenes, vinyl arenes and related compounds. It also relates to methods and uses related thereto.

Terpenes are a class of unsaturated hydrocarbons produced mainly by plants, particularly conifers. Terpenes are further classified by the number of carbons: monoterpenes (C10), sesquiterpenes (C15), diterpenes (C20), etc. (https://en.wikipedia.org/wiki/Terpene). Due to their high volatility and pleasant olfactory properties, terpenes are of high interest for flavors and fragrances industry, but also for food & feed and upon modification for pharmaceutical and fine chemicals industry. Oxyfunctionalization of terpenes, in particular hydroxylation and epoxidation in a stereo-, regio- and enantioselective manner is a reaction often required. Some monoterpenes, like α-pinene, β-pinene, 3-carene, limonene, camphene, terpinolene are already considered as a renewable feedstock available for industrial application.

This work builds on the known properties of plant seed peroxygenases, which have been described to belong to caleosin-like (calcium binding) proteins (J. Biol. Chem. Vol. 281, NO. 44, pp. 33140-33151, Nov. 3, 2006). These are heme-containing proteins which contain an EF-hand calcium-binding motif. These proteins are found in oat microsomes and lipid droplets, they are membrane bound and they catalyze hydroperoxide dependent monooxygenation of unsaturated fatty acids giving as a product fatty acid hydroperoxides. In plants, they form the so-called “the peroxygenase pathway”, together with epoxide hydrolase. See for example US2012/03019323, relating to recombinant oleaginous microorganisms having increased oil content due to the expression of a caleosin polypeptide. Specifically disclosed is the plant caleosin from Arabidopsis thaliana. Blée et al. (FEBS J. 279 (2012), 3981-3995) report on an epoxidization function of the A. thaliana caleosin-like peoxygenase.

Fuchs et al. (J. Mol. Catalysis B: Enzymatic 96 (2013) 52-60) discloses the use of the Solanum lycopersicum caleosin-like peroxygenase for the oxyfunctionalization of terpenes.

Various reports can be found in the literature describing recombinant production of plant caleosins and fungal homologues. However, application of these enzymes at a commercial scale is hampered by several obstacles. A major drawback for improvement and (industrial) application of existing caleosin-like/peroxygenases is the fact that they are either of fungal or plant origin. This requires plant or fungal expression systems, which hampers the production and engineering efforts.

Therefore, the present inventors set out to overcome at least part these drawbacks. More in particular, they set out to identify novel enzymes of non-fungal and non-plant origin and possessing a high substrate promiscuity, i.e. displaying caleosin-like peroxygenase activity against a diverse set of (commercially relevant) substrates. Ideally, the enzyme can be expressed at a high level in a fast growing and easy to manipulate host cells such as a yeast host cell, or more preferably even faster growing and easier to manipulate host cells such as a bacterial host cell.

It was surprisingly found that, using the sequence of Arabidopsis thaliana caleosin (AEE85247.1), several bacterial homologues could be identified which show the desired catalytic (i.e. epoxidation and hydroxylation) activity against various terpenes and other unsaturated molecules. The enzymes can be expressed in and purified from bacteria in a good yield. A facile procedure using detergent was developed for isolating recombinantly produced enzyme from a host cell. Furthermore, using a secretion signal, the yield of recombinant protein could be increased and its localization could be targeted to the membranes and periplasm. Advantageously, the enzymes can be used in isolated form and in the form of whole cells.

The main product of such conversion is an epoxide that can be further hydrolyzed, either spontaneously or using a catalyst (chemical or enzymatic). We envision that herein described enzymes and processes can be used for F&F, cosmetics and food & beverages. Beside those applications, there is a potential of using modified terpenes and unsaturated building blocks as building blocks in the pharmaceutical and polymer industry.

Accordingly, the invention relates to an isolated polypeptide having caleosin-like peroxygenase activity, selected from the group consisting of:
(a) a polypeptide comprising an amino acid sequence having at least 50% pairwise sequence identity when aligned to at least 150 consecutive amino acid residues of Seq. No. 2 (see FIG. 1; Table 1), and comprising at least the following heme-coordinating motifs:
   i) HXXFFD,
   ii) H(X)XD, preferably HXXD, more preferably HXSD, most preferably HGSD, wherein X is any amino acid;
(b) a fragment of the polypeptide of (a) that has caleosin-like peroxygenase activity.

In one embodiment, the invention provides a method for oxyfunctionalization of a substrate of interest, comprising contacting a cyclic or non-cyclic aliphatic alkene or a terpene substrate with a source of hydrogen peroxide and a polypeptide having caleosin-like peroxygenase activity (EC 1.11.2.1), wherein the polypeptide is selected from the group consisting of:
(a) a polypeptide comprising an amino acid sequence having at least 50% pairwise sequence identity when aligned to at least 150 consecutive amino acid residues of a sequence shown in FIG. 1, and comprising at least the following heme-coordinating motifs:
   i) HXXFFD;
   ii) H(X)XD, preferably HXXD, more preferably HXSD, most preferably HGSD; wherein X is any amino acid; and
(b) a fragment of the polypeptide of (a) that has caleosin-like peroxygenase activity.

In a specific aspect, the invention provides a method for oxyfunctionalization of a substrate of interest, comprising contacting the substrate with a source of hydrogen peroxide and a polypeptide having caleosin-like peroxygenase activity (EC 1.11.2.1), wherein the polypeptide is of bacterial origin and selected from the group consisting of:
(a) a polypeptide comprising an amino acid sequence having at least 60% pairwise sequence identity, preferably when aligned to at least 150 consecutive amino acid residues, with any one of the bacterial sequences depicted in Seq. no. 2-10 of FIG. 1, and comprising at least the following heme-coordinating motifs:
i) HXXFFD;
ii) H(X)XD, preferably HXXD, more preferably HXSD,
wherein X is any amino acid; and
(b) a fragment of the polypeptide of (a) that has caleosin-like peroxygenase activity.

The prior art fails to teach or suggest that the bacterial polypeptides identified herein, let alone that they are advantageously used for the oxyfunctionalization of a substrate of interest.

The term “having caleosin-like peroxygenase activity” as used herein refers to the capacity to catalyse the epoxidation of unsaturated fatty acids. Exemplary activities include epoxidation, for example epoxidation of oleic acid.

A polypeptide (fragment) can be readily screened for having caleosin-like peroxygenase activity using assays known in the art. For example, epoxidation of unsaturated fatty acids, such as oleic acid. In addition, a polypeptide showing peroxidase activity can be identified using assays known in the art. For example, oxidation of ABTS (2,2′-azino-bis(3-ethylbenzothiazoline-6-sulfonic acid)), guaiacol and/or 2,6-dimethoxyphenol can be detected (Van Bloois et al. Appl Microbiol Biotechnol. 2010; 86(5): 1419-1430). Preferably, a polypeptide having caleosin-like peroxygenase activity according to the invention displays an in vitro activity of converting at least 15% of 1 mM oleic acid within 4 h at 25° C. when 5 μM enzyme is used.

The term “pairwise sequence identity percentage” generally means the coefficient between amino acid residue positions that have the same amino acid in two aligned sequences over all positions when the two protein sequences are aligned. Percent (%) sequence identity with respect to amino acid sequences disclosed herein is defined as the percentage of amino acid residues in a candidate sequence that are pair-wise identical with the amino acid residues in a reference sequence, i.e. a protein molecule or fragment of the present disclosure, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using public available computer software such as pairwise sequence identity when aligned using the Global alignment with free end gaps method, BLAST, ALIGN, or Megalign (DNASTAR) software. Those skilled in the art can determine appropriate parameters for measuring alignment, including any algorithms needed to achieve maximum alignment over the full length of the sequences being compared.

The term “amino acid” or “amino acid residue” refers to an a- or β-amino carboxylic acid. When used in connection with a protein or peptide, the term “amino acid” or “amino acid residue” typically refers to an a-amino carboxylic acid having its art recognized definition such as an amino acid selected from the group consisting of: L-alanine (Ala or A); L-arginine (Arg or R); L-asparagine (Asn or N); L-aspartic acid (Asp or D); L-cysteine (Cys or C); L-glutamine (Gln or Q); L-glutamic acid (Glu or E); glycine (Gly or G); L-histidine (His or H); L-isoleucine (Ile or I): L-leucine (Leu or L); L-lysine (Lys or K); L-methionine (Met or M); L-phenylalanine (Phe or F); L-proline (Pro or P); L-serine (Ser or S); L-threonine (Thr or T); L-tryptophan (Trp or W); L-tyrosine (Tyr or Y); and L-valine (Val or V), although modified, synthetic, or rare amino acids such as e.g. taurine, ornithine, selenocysteine, homocystine, hydroxyproline, thioproline, iodotyrosine, 3-nitro-tyrosine, ornithine, citrulline, canavanine, 5-hydroxytryptophane, carnosine, cycloleucine, 3,4-dihydroxy phenylalanine, N-acetylcysteine, prolino 1, allylglycine or acetidine-2-carboxylic acid may be used as desired. Generally, amino acids can be grouped as having a nonpolar side chain (e.g., Ala, Cys, Ile, Leu, Met, Phe, Pro, Val); a negatively charged side chain (e.g., Asp, GIu); a positively charged side chain (e.g., Arg, His, Lys); or an uncharged polar side chain (e.g., Asn, Cys, Gln, Gly, His, Met, Phe, Ser, Thr, Trp, and Tyr).

A “fragment” as used herein refers to a portion of a parental protein which portion has peroxygenase activity. Such a fragment can comprise consecutive amino acids of the parental protein. A “fragment” can also refer to a protein in which fragments of a parental protein are fused together. A fragment can also comprise modifications such as amino acid substitutions, amino acid deletions or amino acid insertions compared to the parental protein.

An enzyme for use in the present invention comprises at least the two heme-coordinating motifs HXXFFD (indicated as “Motif 1” in FIG. 1) and H(X)XD (indicated as “Motif 3” in FIG. 1), wherein X is any amino acid.

In one embodiment, at least one of the X residues in motif 1 is selected from the group consisting of V, S and A. Preferred motifs include those containing the sequence VS, AE, VA, SA, VD or VS.

Motif 3 is of the sequence H(X)XD, indicating that the H and D residues can be spaced by either one or two amino acid residues. In one aspect, motif 3 is HXD, wherein X is preferably an acidic residue such as D. Preferably, motif 3 has the sequence HXXD, preferably wherein the spacing X residues are independently selected from G, S, A and D. More preferably, motif 3 is HXSD, most preferably HGSD.

A polypeptide for use in the invention may furthermore contain a calcium binding EF-hand motif. For example, the calcium binding EF-hand motif comprises at least two, preferably all, of the glutamate residues indicated as M2, M4 and M5 in FIG. 1), corresponding to residues E42, E129 and E150 of the amino acid sequence of Seq. no. 2. In one embodiment, glutamate residues corresponding to M2 and M4, M2 and M5, or M4 and M5 are present. In a specific aspect, glutamate residues corresponding to M2, M4 and M5 are present.

In one embodiment, the polypeptide comprises a sequence that has at least 65%, 70%, 75%, 80%, 85%, 90%, 92%, 95%, 96%, 97%, 98% or 99% pairwise sequence identity with any one of Seq. no. 2-10 of FIG. 1, or a fragment thereof having caleosin-like peroxygenase activity.

Preferably, the sequence shows at least 75%, 80%, 85%, 90%, 92%, 95%, 97%, 98% or 99% pairwise sequence identity with any one of Seq. no. 2-10, more preferably with Seq. no. 2 or 3 (T3A1 and T3G1 enzymes), or a fragment thereof that has peroxygenase activity.

A polypeptide for use according to the invention may comprise (by genetic fusion) one or more additional amino acid sequences or protein tag(s) at its N- and/or C-terminus. In one embodiment, the polypeptide comprises an N-terminal tag. In another embodiment, the polypeptide comprises a C-terminal tag. In a further embodiment, the polypeptide comprises both an N- and a C-terminal tag. The additional tag sequence(s) may aid in the expression yield, folding, solubilization, purification and/or immobilization of the polypeptide. Such sequences are well known in the art. Exemplary fusion tags include an (N-terminal) secretion signal sequence, such as a DsbA or Tat signal sequence, a maltose binding protein, N-utilization substance A (NusA), glutathione S-transferase (GST), biotin carboxyl carrier protein, thioredoxin, and cellulose binding domain, short peptide tags such as oligohistidine (6xHis; His-tag), oligolysine, S-peptide, and the FLAG peptide. Exemplary solubility tags include SUMO (Small Ubiquitin-like Modifier) or MBP (maltose-binding protein). In a specific aspect, the enzyme contains an N-terminal His-tag. Alternatively, or additionally, it is provided with a SUMO tag. The tag sequence(s) may be (proteolytically) removed from the polypeptide prior to their application to catalyze a peroxygenase reaction. For example, SUMO fusion proteins can be cleaved to remove the SUMO moiety using SUMO-specific proteases such as Ulp1.

The polypeptide may be used in any suitable format or degree of purification. In one embodiment, the polypeptide is comprised in whole cells or in a cell-free extract. In another embodiment, the polypeptide is used as a (partially) purified, and optionally immobilized, enzyme.

The invention also relates to a composition comprising one or more polypeptide(s) according to the invention. For example, the composition comprises whole cells, permeabilized cells, a cell extract or a cell-free extract comprising a recombinantly expressed enzyme of the invention. In another embodiment, the composition comprises the enzyme(s) in a soluble or immobilized form. The composition may be a reaction mixture comprising one or more peroxygenases, one or more substrates, a source of H₂O₂, and/or products.

Also provided is an isolated polynucleotide encoding a polypeptide according to the invention. The polynucleotide may be comprised in a nucleic acid construct or expression vector, preferably wherein the polynucleotide is operably linked to one or more control sequence(s) that direct the production of the polypeptide in an expression host. Exemplary expression vectors are known in the art. The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like. In one embodiment, the vector is an E. coli expression vector. For example, polypeptides can be expressed using a pET-based (IPTG-inducible) vector or a pBAD-based (arabinose inducible) vector.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell. The control sequence may also be a leader, a non-translated region of an mRNA that is important for translation by the host cell. The leader is operably linked to the 5′-terminus of the polynucleotide encoding the polypeptide. Any leader that is functional in the host cell may be used. The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention. Preferred terminators for bacterial host cells are obtained from the genes for Bacillus clausii alkaline protease {aprH), Bacillus licheniformis alpha-amylase (amyL), and Escherichia coli ribosomal RNA (rrnB). The control sequence may also be an mRNA stabilizer region downstream of a promoter and upstream of the coding sequence of a gene which increases expression of the gene. Examples of suitable mRNA stabilizer regions are obtained from a Bacillus thuringiensis crylllA gene and a Bacillus subtilis SP82 gene.

A further embodiment of the invention relates to a recombinant host cell comprising the nucleic acid construct or expression vector of the invention encoding a polypeptide as herein disclosed. In one aspect, the encoding nucleic acid sequence is part of an expression vector. In another embodiment, the encoding nucleic acid sequence is integrated in the genome of the host cell. For example, it is possible to integrate the encoding gene into the genome of a host organism by methods known in the art, including genome editing methods, homologous recombination, and methods involving the CRISPR Cas system.

The host cell may be any cell useful in the recombinant production of a polypeptide of the present invention, e.g. a prokaryote or a eukaryote. For example, the host cell is a bacterial host cell or a fungal host cell. The prokaryotic host cell may be any Gram-positive or Gram-negative bacterium. Gram-positive bacteria include Bacillus, Brevibacillus, Clostridium, Enterococcus, Geobacillus, Lactobacillus, Lactococcus, Oceanobacillus, Paenibacillus, Staphylococcus, Streptococcus, and Streptomyces. Gram-negative bacteria include Campylobacter, E. coli, Flavobacterium, Fusobacterium, Helicobacter, Ilyobacter, Neisseria, Pseudomonas, Salmonella, Paracoccus and Ureaplasma.

In a specific aspect, the host cell is E. coli. For expression with pET-based vectors E. coli BL21, E. coli C41/C43 or E. coli BL21AI strains can be used, while for pBAD-based vectors E. coli NEB108, E. coli TOP10, E. coli BL21AI and other standard strains be used.

The recombinant bacterial host may be any Bacillales, including Bacillus amyloliquefaciens, Brevibacillus brevis, Bacillus circulans, Bacillus clausii, Bacillus coagulans, Bacillus lentus, Bacillus licheniformis, Geobacillus stearothermophilus, Bacillus subtilis, and Bacillus thuringiensis. The recombinant bacterial host may also be any Streptomyces including Streptomyces achromogenes, Streptomyces avermitilis, Streptomyces coelicolor, Streptomyces griseus, and Streptomyces lividans. The recombinant bacterial host may also be any Paracoccus including Paracoccus denitrificans, Paracoccus versutus, Paracoccus carotinifaciens, Paracoccus marcusii and Paracoccus zeaxanthinifaciens.

In another embodiment, the host cell is a fungal host cell, preferably wherein the recombinant fungal host cell is a member of a genus selected from the group consisting of: Aspergillus, Blakeslea, Botrytis, Candida, Cercospora, Cryptococcus, Cunninghamella, Fusarium (Gibberella), Kluyveromyces, Lipomyces, Mortierella, Mucor, Neurospora, Penicillium, Phycomyces, Pichia (Hansenula), Puccinia, Pythium, Rhodosporidium, Rhodotorula, Saccharomyces, Sclerotium, Trichoderma, Trichosporon, Xanthophyllomyces (Phaffia), and Yarrowia, or is of a species selected from the group consisting of: Aspergillus terreus, Aspergillus nidulans, Aspergillus niger, Blakeslea trispora, Botrytis cinerea, Candida japonica, Candida pulcherrima, Candida revkaufi, Candida tropicalis, Candida utilis, Cercospora nicotianae, Cryptococcus curvatus, Cunninghamella echinulata, Cunninghamella elegans, Fusarium fujikuroi (Gibberella zeae), Kluyveromyces lactis, Lipomyces starkeyi, Lipomyces lipoferus, Mortierella alpina, Mortierella ramanniana, Mortierella isabellina, Mortierella vinacea, Mucor circinelloides, Neurospora crassa, Phycomyces blakesleanus, Pichia pastoris, Puccinia distincta, Pythium irregulare, Rhodosporidium toruloides, Rhodotorula glutinis, Rhodotorula graminis, Rhodotorula mucilaginosa, Rhodotorula pinicola, Rhodotorula gracilis, Saccharomyces cerevisiae, Sclerotium rolfsii, Trichoderma reesei, Trichosporon cutaneum, Trichosporon pullulans, Xanthophyllomyces dendrorhous (Phaffia rhodozyma), and Yarrowia lipolytica.

Host cells may be genetically modified to have characteristics that improve genetic manipulation, protein secretion, protein stability and/or other properties desirable for expression or secretion of a peroxygenase enzyme. For example, host cells may be modified to contain an enzyme capable of removing a tag sequence that is fused to a polypeptide of the invention. For example, the host cell comprises a vector that encodes not only a SUMO- and His-tagged peroxygenase of interest, but also SUMO-tagged Ulp1 protease. Co-expression of these two proteins results in the in vivo cleavage of the enzyme of interest from the SUMO tag, while still leaving the enzyme of interest in a form that can be purified from a soluble cell lysate by nickel affinity chromatography.

Also provided is a method of producing a polypeptide having caleosin-like peroxygenase activity, comprising (a) cultivating said host cell under conditions conducive for production of the polypeptide; and (b) recovering the polypeptide. Suitable media for growing the host of the invention are well known in the art, for example, see Sambrook et al., Molecular Cloning (1989), supra. In general, a suitable medium contains all the essential nutrients for the growth of the host system. The medium can be supplemented with antibiotics that are selected for host-vector system. The medium can be supplemented with 5-aminolevulinic acid (5-ALA) to improve heme synthesis, or hemin (ferric chloride heme) can be added to the medium. In this way the amount of holo-enzyme can be improved.

In one aspect, the invention provides a method of producing a polypeptide having caleosin-like peroxygenase activity, comprising:
(a) cultivating a host cell expressing a polypeptide of the invention under conditions conducive for production of the polypeptide;
(b) preparing from the host cell a fraction comprising membrane-associated proteins;
(c) solubilizing said membrane-associated proteins using a detergent and
(d) recovering the polypeptide from the solubilized fraction (supernatant).

Also provided herein is a bacterial host cell fraction comprising a recombinant membrane-associated polypeptide having peroxygenase activity obtainable by steps (a) and (b) of the above method.

Thus, an expressed polypeptide can be used in the form of whole cells, permeabilized cells, a cell extract or a cell-free extract comprising an enzyme of the invention. In another embodiment, the enzyme is used in a soluble or immobilized form. Expressed enzyme(s) may be recovered from cells using methods known in the art. Optionally, a protein can be enriched for (e.g., purified or partially purified) using methods well known in the art. For example, the polypeptide may be isolated by conventional procedures including centrifugation, filtration, extraction, spray-drying, evaporation, chromatography (e.g., ion exchange, solid phase binding, affinity, hydrophobic interaction, chromatofocusing, and size exclusion chromatography) and/or filtration, or precipitation. Protein refolding steps can be used, as desired, in completing the configuration of the mature protein. Finally, high performance liquid chromatography (HPLC) can be employed in the final purification steps.

As will be appreciated by a person skilled in the art, a polypeptide as herein disclosed finds its application in converting a broad range of substrates into desirable products. In one embodiment, the substrate is an aliphatic alkene, a vinyl arene or a terpene.

For example, the invention provides a method for oxyfunctionalization of a cyclic or non-cyclic (i.e. linear) aliphatic alkene or vinyl arene substrate, comprising contacting the substrate with a source of hydrogen peroxide and a polypeptide having caleosin-like peroxygenase activity. The aliphatic alkene or vinyl arene may be unsubstituted or substituted. For example, the aliphatic alkene substrate has one or more substituents selected from the group consisting of halogen, hydroxyl, carboxyl, amino, nitro, cyano, thiol, sulphonyl, formyl, acetyl, methoxy, ethoxy, carbamoyl and sulfamoyl. Preferably, the substituent(s) are selected from the group consisting of chloro, hydroxyl, carboxyl and sulphonyl; in particular chloro and carboxyl. Suitable substrates include aliphatic alkenes which contain at least three carbon atoms, and have a carbon-carbon double bond, for example a C═C bond at one end.

Exemplary non-cyclic aliphatic alkene substrates include propene, butene, pentene, hexene, heptene, octene, nonene, decene, undecene, dodecene, tridecene, tetradecene, pentadecene, or hexadecene, or an isomer thereof. In one aspect, the aliphatic alkene substrate is selected from the group consisting of propene, 1-butene, 1-pentene, 1-hexene, 2-hexene, 3-hexene, 1-heptene, 1-octene, 2-methyl-2-butene, 2,3-dimethyl-2-butene, cis/trans-2-butene, isobutene, 1,3-butadiene, 2-, 3- and 4-octene, oleic acid, and isomers thereof.

Exemplary cyclic aliphatic alkene substrates include C3-C12 cycloalkenes, such as cyclopropene, cyclobutene, cyclopentene, cyclohexene, cycloheptene, and cyclooctene.

Exemplary vinyl arene substrates include styrene, β-methylstyrene, indene and stilbene.

In one aspect, the method comprises the oxidation of styrene to styrene epoxide and/or styrene aldehyde. In a specific aspect, an enzyme provided herein is used as catalyst in an oxygenation reaction, giving rise to an aldehyde as a direct anti-markovnikov peroxygenase product, i.e. not as a secondary product resulting from rearrangement of an epoxide. For example, styrene is directly converted to its aldehyde using polypeptide T3G1, T3A1, T3A2, or a functional fragment thereof.

In another embodiment, the invention provides a method for oxyfunctionalization of a terpene substrate of interest, comprising contacting the terpene substrate with a source of hydrogen peroxide and a polypeptide having caleosin-like peroxygenase activity as herein disclosed. The terpene substrate can be isoprene or a monoterpene. In one aspect, the terpene is a cyclic terpene, preferably a monocyclic monoterpene, such as limonene. For example, provided is a method for the conversion of (+)-limonene to cis-limonene epoxide.

Still further, the invention relates to a method for preparing a substituted or unsubstituted indigo dye, comprising contacting, preferably at a pH in the range of 6-9, a substituted or unsubstituted indole with a source of hydrogen peroxide and a polypeptide as herein defined. Preferably, the polypeptide is T3A1, T3G1, or a functional fragment thereof.

Also encompassed is a method for the degradation (and thus in most cases decolorization) of a textile dye, preferably a vinyl sulfone azo dye, more preferably Reactive blue 19 (RB 19), comprising contacting the dye, preferably at a pH in the range of 3-6, with a source of hydrogen peroxide and a polypeptide according to the invention, in particular wherein the polypeptide is T3G1, T3A1, T3A2, or a functional fragment thereof.

The hydrogen peroxide required by the caleosin-like peroxygenase may be provided as an aqueous solution of hydrogen peroxide or a hydrogen peroxide precursor for in situ production of hydrogen peroxide. Any solid entity which liberates upon dissolution a peroxide, which is usable by peroxygenase, can serve as a source of hydrogen peroxide. Compounds which yield hydrogen peroxide upon dissolution in water or an appropriate aqueous based medium include metal peroxides, percarbonates, persulphates, perphosphates, peroxyacids, alkyperoxides, acylperoxides, peroxyesters, urea peroxide, perborates and peroxycarboxylic acids or salts thereof.

An alternative source of hydrogen peroxide is a hydrogen peroxide generating enzyme system, such as an oxidase together with a substrate for the oxidase. Examples of combinations of oxidase and substrate comprise, but are not limited to, amino acid oxidase (see e.g. U.S. Pat. No. 6,248,575) and a suitable amino acid, glucose oxidase (see e.g. WO95/29996) and glucose, lactate oxidase and lactate, galactose oxidase (see e.g. WO00/50606) and galactose, formate oxidase and formate (Willot et al.; 2020, ChemCatChem Volume12, Issue10, pp. 2713-2716) and aldose oxidase (see e.g. WO99/31990) and a suitable aldose.

Hydrogen peroxide or a source of hydrogen peroxide may be added at the beginning of or during a method of the invention, e.g. as one or more separate additions of hydrogen peroxide; or continuously as fed-batch addition. Typical amounts of hydrogen peroxide correspond to levels of from 0.001 mM to 25 mM, preferably to levels of from 0.005 mM to 5 mM, and particularly to levels of from 0.01 to 1 mM or 0.02 to 2 mM hydrogen peroxide. Hydrogen peroxide may also be used in an amount corresponding to levels of from 0.1 mM to 25 mM, preferably to levels of from 0.5 mM to 15 mM, more preferably to levels of from 1 mM to 10 mM, and most preferably to levels of from 2 mM to 8 mM hydrogen peroxide. The method of the invention may be carried out with an immobilized peroxygenase.

Herewith, the invention also relates to the use of a polypeptide according to the invention as a catalyst, preferably as a catalyst of a caleosin-like peroxygenase reaction.

A method of the invention may be carried out in an aqueous solvent or buffered system (reaction medium). Suitable buffered systems are easily recognized by one skilled in the art, and include K-phosphate (K—Pi) buffers and Tris.HCl buffers.

The methods according to the invention may be carried out at a temperature between 0 and 90° C., preferably between 5 and 80° C., more preferably between 10 and 70° C., even more preferably between 15 and 60° C., most preferably between 20 and 50° C., and in particular between 20 and 40° C. The methods of the invention may employ a treatment time of from 10 seconds to (at least) 24 hours, preferably from 1 minute to (at least) 12 hours, more preferably from 5 minutes to (at least) 6 hours, most preferably from 5 minutes to (at least) 3 hours, and in particular, from 5 minutes to (at least) 1 hour.

### LEGEND TO THE FIGURES

FIG. 1: Amino acid sequence alignment of Arabidopsis thaliana caleosin (No. 1) and nine newly discovered and characterized bacterial homologues (No. 2-10): T3G1-gb|NDD31306.1, T3A1-tpg|HHO53497.1, T3B1-ref|WP_146069755.1, T3C1-ref|WP_141736382.1, T3D1-ref|WP_104985314.1, T3E1-gb|TPW18992.1, T3F1-gb|PIQ25853.1, T3H1-gb|KYF87516.1, T3A2-gb|KAB2893313.1. Conserved sequence motifs are indicated on top.

FIG. 2: Substrate screening for 9 purified enzymes. Upper four rows correspond to substrates tested in K-acetate buffer pH 4 and lower four rows correspond to the substrates tested in K-phosphate buffer pH 7. The following substrates were tested: KI—potassium iodide; ABTS—2,2′-azino-bis(3-ethylbenzothiazoline-6-sulfonic acid); 2,6-DMP-2,6-dimethoxy-phenol; RB19—reactive blue 19; indole; m-cresol; in.carmine—indigo carmine. Column CTRL is a control sample, without addition of enzyme (buffer and substrate only). The reaction was initiated by addition of H₂O₂, final concentration 2 mM.

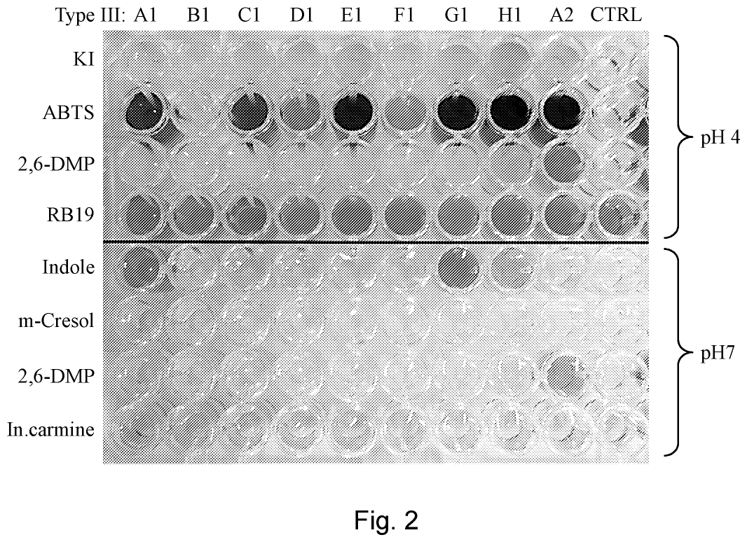

FIG. 3. The UV-Vis spectra of the SUMO-T3G1 purified from the cell-free extract with Rz=0.7 (panel A) and from the Triton X-100 assisted extraction of the cell debris pellet with Rz=2.4 (panel B). The Rz value is the ratio between the absorbance of Soret band (˜405 nm) and the absorbance at 280 nm, used to estimate heme loading.

### EXPERIMENTAL SECTION

### Example 1: Cloning, Expression and Purification of Novel Caleosin-Like Enzymes

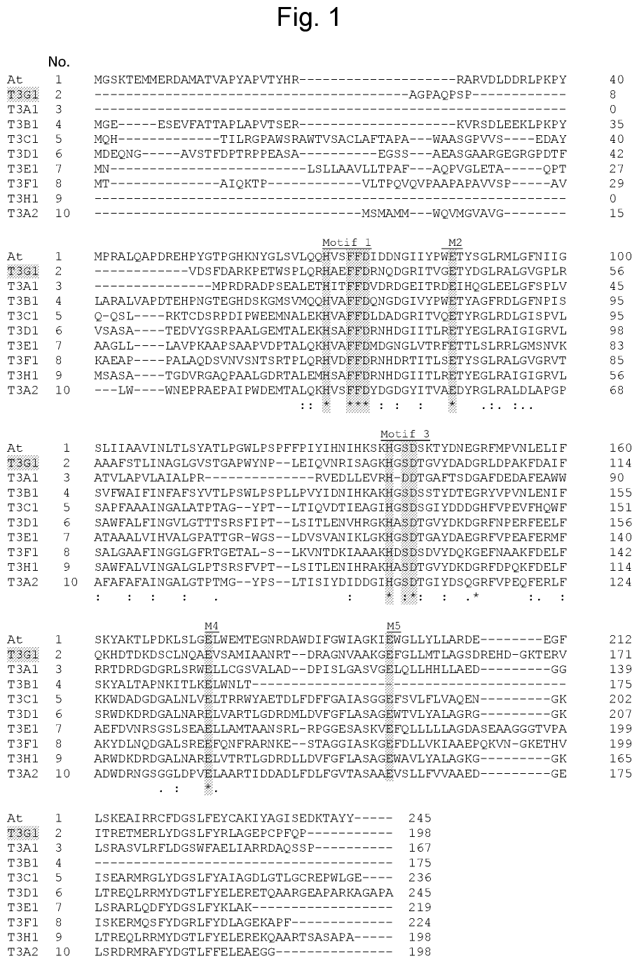

Using the sequence of Arabidopsis thaliana caleosin (AEE85247.1) we identified several bacterial homologues which contain conserved motifs H—X—X—F—F-D and H—(X)—X-D which provide two histidine residues most likely interacting directly with the heme cofactor and forming the active site. Apart from these two motifs (Motif 1 and 3 in FIG. 1) there are also conserved glutamate (E) residues (Motifs M2, M4 and M5) probably acting as a calcium-binding site.

The synthetic genes for 9 bacterial homologues (see Table 1) were cloned in pBAD vector to encode fusion proteins with SUMO peptide. The constructs were transformed in E. coli NEB108, which was used for expression under standard conditions. Briefly, the expression was performed in TB medium supplemented with ampicillin, 5-aminolevulinic acid and 0.02% arabinose. Expression was carried out at 30° C. for 16 h. Harvested cells were disrupted by sonication and the extract was further processed according to a standard procedure for purification using Immobilized Metal Chelate Affinity Chromatography (IMAC).

TABLE 1
Bacterial enzymes having caleosin-like peroxygenase activity for use in the invention.
T3G1 - gb | NDD31306.1, T3A1 - tpg | HHO53497.1, T3B1 - ref | WP_146069755.1, T3C1 - ref | WP_141736382.1,
T3D1 - ref | WP_104985314.1, T3E1 - gb | TPW18992.1, T3F1 - gb | PIQ25853.1, T3H1 - gb | KYF87516.1,
T3A2 - gb | KAB2893313.1.

| Seq. | | NCBI accession | | |
|---|---|---|---|---|
| No. | Name | number | source | Amino acid sequence |
| 2 | T3G1 | NDD31306.1 | Proteobacteria | agpaqpspvd sfdarkpetw splqrhaeff drnqdgritv getydglral gvgplraaaf |
| | | | bacterium | stlinaglgv stgapwynpl eiqvnrisag khgsdtgvyd adgrldpakf daifqkhdtd |
| | | | | kdsclnqaev samiaanrtd ragnvaakge fgllmtlags drehdgkter vitretmerl |
| | | | | ydgslfyrla gepcpfqp |
| 3 | T3A1 | HHO53497.1 | Deltaproteobacteria | mprdradpse alethitffd vdrdgeitrd eihqgleelg fsplvatvla pvlaialprr |
| | | | bacterium | vedllevrhd dtgaftsdga fdedafeaww rrtdrdgdgr lsrwellcgs valaddpisl |
| | | | | gasvgelqll hhllaedggl srasvlrfld gswfaeliar rdaqssp |
| 4 | T3B1 | WP_146069755.1 | Arthrobacter | mgeesevfat taplapvtse rkvrsdleek lpkpylaral vapdtehpng teghdskgms |
| | | | sp. B0490 | vmqqhvaffd qngdgivypw etyagfrdlg fnpissvfwa ifinfafsyv tlpswlpspl |
| | | | | lpvyidnihk akhgsdssty dtegryvpvn lenifskyal tapnkitlke lwnlt |
| 5 | T3C1 | WP_141736382.1 | Oligoflexus | mqhtilrgpa wsrawtvsac laftapawaa sgpvvseday qqslrktcds rpdipweemn |
| | | | tunisiensis | alekhvaffd ldadgritvq etyrglrdlg ispvlsapfa aaingalatp tagyptltiq |
| | | | | vdtieagihg sdsgiydddg hfvpevfhqw fkkwdadgdg alnlveltrr wyaetdlfdf |
| | | | | fgaiasggef svlflvagen gkisearmrg lydgslfyai agdlgtlgcr epwlge |
| 6 | T3D1 | WP_104985314.1 | Sorangium | mdeqngavst fdptrppeas aegssaeasg aargegrgpd tfvsasated vygsrpaalg |
| | | | cellulosum | emtalekhsa ffdrnhdgii tlretyeglr aigigrvlsa wfalfingvl gtttsrsfip |
| | | | | tlsitlenvh rgkhasdtgv ydkdgrfnpe rfeelfsrwd kdrdgalnar elvartlgdr dmldvfgfla |
| | | | | sagewtvlya lagrggkltr eqlrrmydgt lfyeleretq aargeapark |
| | | | | agapa |
| 7 | T3E1 | TPW18992.1 | Elusimicrobia | mnlsllaavl ltpafaqpvg letaqptaag lllavpkaap saapvdptal qkhvaffdmd |
| | | | bacterium | gnglvtrfet tlslrrlgms nvkataaalv ihvalgpatt grwgsldvsv aniklgkhgs |
| | | | | dtgaydaegr fvpeafermf aefdvnrsgs lseaellamt aansrlrpgg esaskvefql |
| | | | | llllagdase aagggtvpal srarlqdfyd gslfyklak |
| 8 | T3F1 | PIQ25853.1 | Candidatus | mtaiqktpvl tpqvqvpaap apavvspavk aeappalaqd svnvsntsrt plqrhvdffd |
| | | | Blackallbacteria | rnhdrtitls etysglralg vgrvtsalga afingglgfr tgetalslkv ntdkiaaakh |
| | | | | dsdsdvydqk gefnaakfde lfakydlnqd galsreefqn frarnkesta ggiaskgefd |
| | | | | llvkiaaepq kvngkethvi skermqsfyd grlfydlage kapf |
| 9 | T3H1 | KYF87516.1 | Sorangium | msasatgdvr gaqpaalgdr talemhsaff drnhdgiitl retyeglrai gigrvlsawf |
| | | | cellulosum | alvingalgl ptsrsfvptl sitlenihra khasdtgvyd kdgrfdpqkf delfarwdkd |
| | | | | rdgalnarel vtrtlgdrdl ldvfgflasa gewavlyala gkggkltreq lrrmydgtlf |
| | | | | yelerekqaa rtsasapa |
| 10 | T3A2 | KAB2893313.1 | Kofleriaceae | msmammwqvm gvavglwwne praepaipwd emtalqkhvs ffdydgdgyi tvaedyrglr |
| | | | bacterium | aldlapgpaf afafaingal gtptmgypsl tisiydiddg ihgsdtgiyd sqgrfvpeqf erlfadwdrn |
| | | | | gsggldpvel aartiddadl fdlfgvtasa aevsllfvva aedgelsrdr mrafydgtlf feleaegg |

### Example 2: Qualitative Analysis of Caleosin-Like Peroxygenase Activity

Initial screening against a panel of common peroxidase substrates showed that the enzymes are produced in the active form. All of them, except T3B1, showed activity against ABTS at pH 4, and some of them were able to oxidize 2,6-DMP at pH 4 and pH 7 (T3A1, T3C1, T3E1, T3G1, T3H1 and T3A2). This information already showed interesting features as compared to the known bacterial peroxidases (DyP-peroxidases), which are limited to being active only at the lower pH. Furthermore, these new enzymes are able to perform dye decolorization/oxidation, as shown using RB19 as a substrate. Then, a very interesting feature was detected, the ability to produce blue color from indole, which corresponds to indigo formation. This is directly attributed to the ability of an enzyme to catalyze oxygen insertion. This prompted us to test the representative enzyme T3G1 against a large panel of various substrates. The results of this screening are summarized in Table 2.

TABLE 2
List of substrates for which the conversion was confirmed and
measured. Reaction mixtures contained 1 mM substrate, 2 mM H₂O₂
and 7 μM enzyme (T3G1, 5 + 2 μM). Reactions were started by
adding H₂O₂ up to 1 mM (final concentration) and 5 μM
enzyme; after 90 min another aliquot of H₂O₂ was added
to bring the H₂O₂ concentration to 2 mM, as well as
another aliquot of T3G1 corresponding to 2 μM, in total
adding 7 μM enzyme.

| No. | Substrate | CAS | % conversion | |
|---|---|---|---|---|
| 1 | Wieland-miescher ketone | 20007-72-1 | 10 | |
| 2 | (S)-Hajos-Wiechert ketone | 17553-86-5 | trace | |
| 3 | (+)-limonene | 5989-27-5 | 81 | (97.3% cis) |
| 4 | (−)-carveol | 99-48-9 | 4 | |
| 5 | 7,8-dihydro-α-ionone | 31499-72-6 | 50 | |
| 6 | trans-β-ionone | 79-77-6 | 48 | |
| 7 | α-ionol | 25312-34-9 | 61 | |
| 8 | α-ionone | 127-41-3 | 61 | |
| 9 | styrene | 100-42-5 | 61 | (17% ee) |
| 10 | α-damascone | 43052-87-5 | 26 | |
| 11 | δ-damascone | 57378-68-4 | 16 | |
| 12 | propanolol | 318-98-9 | 5 | |
| 13 | β-methyl-styrene | 637-50-3 | 86 | |
| 14 | valencene | 4630-07-3 | 3 | |
| 15 | creosol | 93-51-6 | trace | |
| 16 | trans-stilbene | 103-30-0 | 17 | |
| 17 | 1-decene | 872-05-9 | 13 | |
| 18 | indene | 95-13-6 | 27 | |
| 19 | trans-3-hexene | 13269-52-8 | 40 | |
| 20 | cis-2-heptene | 6443-92-1 | 81 | |
| 21 | ethylbenzene | 100-41-4 | trace | |
| 22 | oleic acid | 112-80-1 | 19 | |
| 23 | cis-cyclooctene | 931-87-3 | 83 | |
| 24 | terpinene | 99-86-5 | 73 | |
| 25 | thioanisole | 100-68-5 | 40 | (74% ee) |

| | | | |
|---|---|---|---|
| 26 | cyclohexene | 110-83-8 | 32 |
| 27 | 1-hexene | 592-41-6 | trace |
| 28 | 1-heptene | 592-76-7 | trace |
| 29 | 2-methyl-1-butene | 563-46-2 | trace |
| 30 | 2-methyl-1,3-butadiene | 78-79-5 | trace |
| 31 | trans-2-hexene | 4050-45-7 | trace |
| 32 | cis-2-hexene | 7688-21-3 | trace |
| 33 | 1-octene | 111-66-0 | 65 |
| 34 | trans-2-octene | 13389-42-9 | 99 |
| 35 | trans-3-octene | 14919-01-8 | 99 |
| 36 | trans-4-octene | 14850-23-8 | 99 |
| 37 | 1,9-decadiene | 1647-16-1 | 15 (13% double |
| | | | epoxide) |

a) “Trace” indicates that a peak is observed for the expected mass which is not present in the control sample, which peak is below the quantification limit.

### Detectable Product

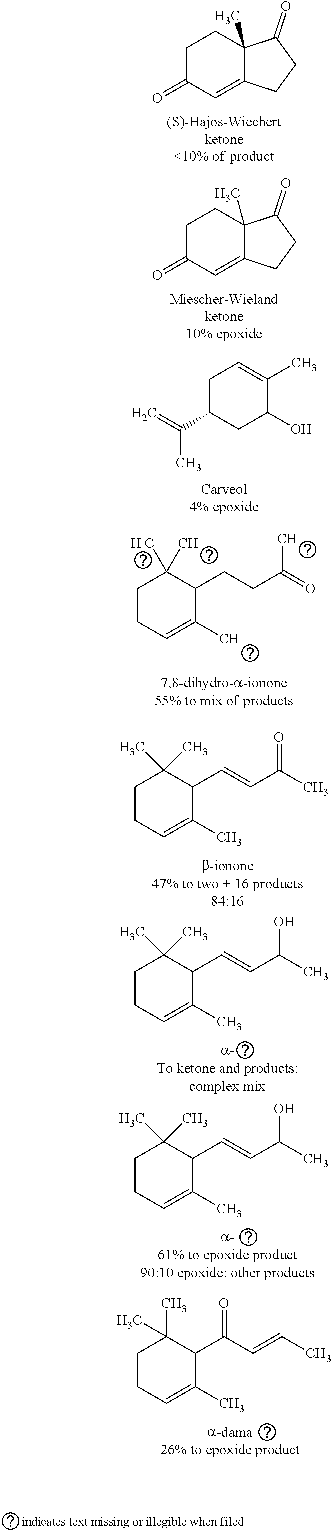

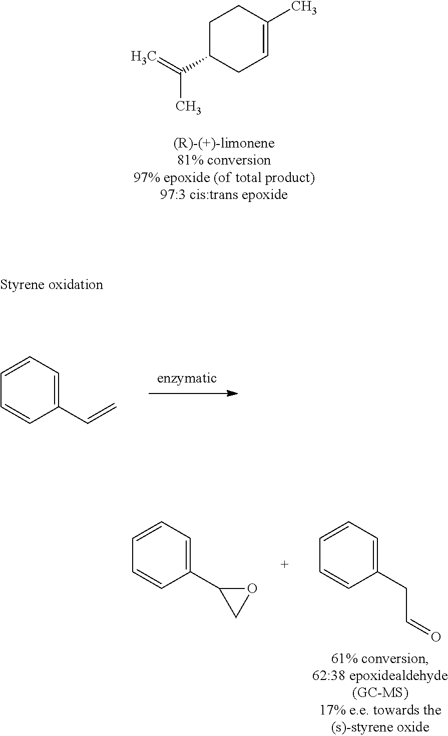

### Example 3: Development of Facile Purification Method

Polypeptides T3A1 and T3G1, representing active and highly expressed enzymes (according to the small-scale trials), were expressed again on 500 ml TB 0.5 mM 5-ALA, 0.02% arabinose at 30° C. overnight and purification was attempted from a clarified cell free extract. For T3A1, a large amount of protein was obtained but with low heme loading. For T3G1, a small amount of heme-loaded enzyme was obtained. A strong red color was observed in the pellet after clarification of CFE. Considering that the plant homologues are known membrane-associated proteins, this suggested that most of the bacterial recombinant protein is localized in the membrane fraction. The pellet was resuspended in 1% Triton X-100 in buffer A (50 mM K-phosphate buffer pH 7.5 with 150 mM NaCl) and incubated on ice for 20 min, then spun down at 12000 rpm for 1 h.

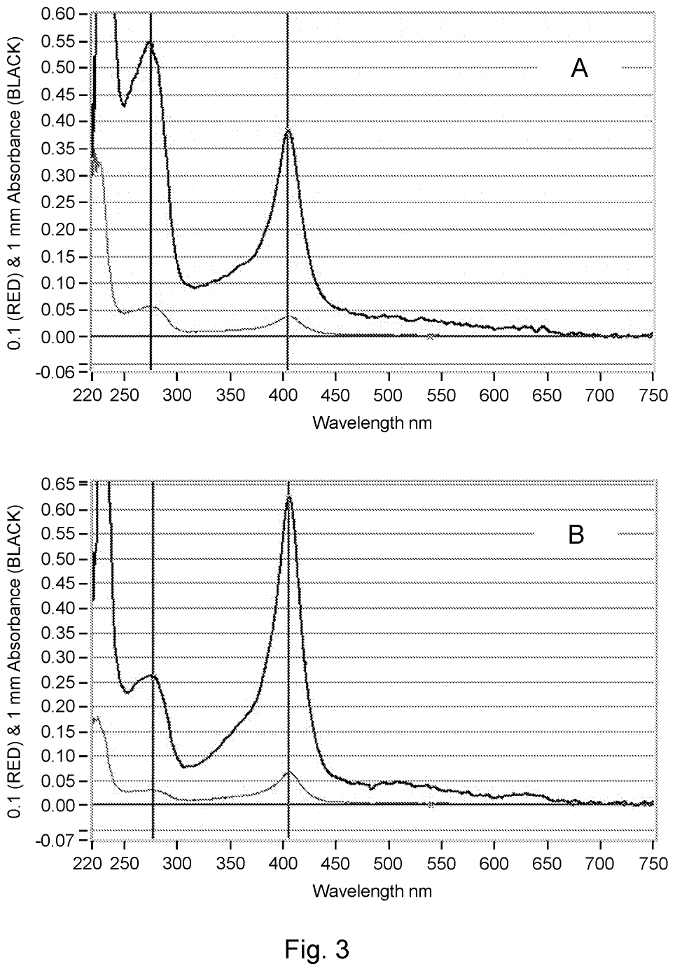

The supernatant obtained had an intense red color whereas the pellet became yellow/brown. The enzyme was purified using IMAC chromatography on Ni-Sepharose resin using protocol known in art and had intense red color and increased Rz value. The Rz value is a ratio between the absorbance of Soret band (˜405 nm) and the absorbance at 280 nm, used for indication of heme loading. See the UV-Vis spectra of FIG. 3,

In conclusion, using this simple method we were able to obtain high yield of fully loaded, active enzyme, without the usual hurdles described for the membrane-associated enzymes.

The ThermoFluor method can be used to measure apparent melting temperature of a protein. This experiment shows T_{m app} (T3G1)=61° C., which puts T3G1 in a moderately stable enzyme.

### Example 4: Optimizing the System for the Whole Cell Conversion

Having in mind that terpenes epoxidation/hydroxylation is a reaction of interest for various uses, and that terpenes are volatile compounds, we looked into the possibility of using whole cells for the conversion. For this purpose, the expression was carried out as usual and cells were pelleted from the 5 ml culture of the induced culture and a control E. coli culture. Then, the cells were resuspended in a K-phosphate buffer pH 7, substrate was added and 1 mM H₂O₂ was added. After 1 h, another aliquot of H₂O₂ was added and the reaction was incubated for another hour. Then, the reaction was terminated by extraction using ethyl-acetate and the sample was analyzed using GC-MS.

After having confirmed product formation, an attempt was made to improve the localization of the enzyme in the membrane and periplasm of bacteria. This was done by recloning T3G1 into a pBAD vector as a fusion protein with a DsbA signal sequence. Expression trials showed an increase in the yield of the produced protein.

1. A method for oxyfunctionalization of a substrate of interest, comprising contacting the substrate with a source of hydrogen peroxide and a polypeptide having caleosin-like peroxygenase activity (EC 1.11.2.1), wherein the polypeptide is of bacterial origin and selected from the group consisting of:
(a) a polypeptide comprising an amino acid sequence having at least 60% pairwise sequence identity with any one of Seq. no. 2-10 of FIG. 1, and comprising at least the following heme-coordinating motifs:
i) HXXFFD;
ii) H(X)XD, preferably HXXD, more preferably HXSD,
wherein X is any amino acid; and
(b) a fragment of the polypeptide of (a) that has caleosin-like peroxygenase activity.

2. The method of claim 1, wherein the polypeptide comprises a calcium binding EF-hand motif.

3. The method of claim 2, wherein the calcium binding EF-hand motif comprises two or more glutamate residues corresponding to E42, E129 and E150 of the amino acid sequence of Seq. no. 2 as shown in Table 2.

4. The method according to claim 1, wherein the polypeptide comprises a sequence that has at least 70% pairwise sequence identity with any one of Seq. no. 2-10 of FIG. 1, or a fragment thereof that has caleosin-like peroxygenase activity.

5. The method according to claim 1, wherein the polypeptide comprises a sequence of Seq. no. 2 or 3, or a fragment thereof that has peroxygenase activity.

6. The method according to claim 1, wherein the polypeptide further comprises an N- and/or C-terminal protein tag allowing for enhanced expression, solubilization, purification, targeting, secretion and/or immobilization.

7. The method according to claim 1, wherein the polypeptide is comprised in whole cells or in a cell-free extract, or wherein the polypeptide is used as purified, and optionally immobilized, enzyme.

8. The method according to claim 1, wherein the substrate is an aliphatic alkene, a vinyl arene or a terpene.

9. The method of claim 8, wherein the aliphatic alkene substrate has one or more substituents selected from the group consisting of halogen, hydroxyl, carboxyl, amino, nitro, cyano, thiol, sulphonyl, formyl, acetyl, methoxy, ethoxy, carbamoyl and sulfamoyl.

10. The method of claim 9, wherein the substituent(s) are selected from the group consisting of chloro, hydroxyl, carboxyl and sulphonyl; in particular chloro and carboxyl.

11. The method of claim 8, wherein the aliphatic alkene contains at least three carbon atoms, and has a carbon-carbon double bond at one end.

12. The method of claim 8, wherein the aliphatic alkene substrate is a non-cyclic aliphatic alkene.

13. The method of claim 8, wherein the aliphatic alkene substrate is a cyclic aliphatic alkene.

14. The method of claim 8, wherein the vinyl arene substrate is styrene, β-methylstyrene, indene or stilbene.

15. The method of claim 8, wherein the terpene substrate is isoprene or a monoterpene; preferably wherein the terpene is a cyclic terpene, more preferably a monocyclic monoterpene, such as limonene.

16. A method for preparing a substituted or unsubstituted indigo dye, comprising contacting a substituted or unsubstituted indole with a source of hydrogen peroxide and a polypeptide of claim 1.

17. The method of claim 1 wherein the polypeptide is a biocatalyst, or a catalyst for oxyfunctionalization.

18. A nucleic acid construct or expression vector comprising a polynucleotide sequence encoding the polypeptide of claim 1, the polynucleotide sequence being operably linked to one or more control sequence(s) that direct the production of the polypeptide in a bacterial or fungal expression host.

19. A recombinant host cell that is a bacterial or fungal host cell, comprising the nucleic acid construct or expression vector of claim 18.

20. A method of producing a polypeptide having caleosin-like peroxygenase activity, comprising:
(a) cultivating the host cell of claim 19 under conditions conducive for production of the polypeptide;
(b) preparing from the host cell a fraction comprising membrane-associated proteins;
(c) solubilizing said membrane-associated proteins using a detergent and (d) recovering the polypeptide from the solubilized fraction (supernatant).

21. The method of claim 12 wherein the non-cyclic aliphatic alkene is selected from the group consisting of propene, butene, pentene, hexene, heptene, octene, nonene, decene, undecene, dodecene, tridecene, tetradecene, pentadecene, or hexadecene, and isomers thereof.

22. The method of claim 13 wherein the cyclic aliphatic alkene is selected from the group consisting of cyclopropene, cyclobutene, cyclopentene, cyclohexene, cycloheptene and cyclooctene.

23. The method of claim 16 wherein the polypeptide comprises a sequence of SEQ ID NO: 2 or SEQ ID NO: 3, or a fragment thereof that has peroxygenase activity.

24. The method of claim 17 wherein the catalyst is for epoxidation of an aliphatic alkene, a vinyl arene or a terpene substrate.
